(19) United States
(12) Patent Application Publication
(10) Pub. No.: US 2022/0319720 A1
(43) Pub. Date: Oct. 6, 2022

(54) SYSTEMS AND METHODS FOR CARE AND DISEASE MANAGEMENT

(71) Applicant: P3 Health Partners, Henderson, NV (US)

(72) Inventors: Sarah BUSSMANN, Las Vegas, NV (US); Ajay CHAUDHARY, Cerritos, CA (US); Unmesh SRIVASTAVA, Monrovia, CA (US)

(73) Assignee: P3 Health Partners, Henderson, NV (US)

(21) Appl. No.: 17/657,183

(22) Filed: Mar. 30, 2022

Related U.S. Application Data
(60) Provisional application No. 63/169,364, filed on Apr. 1, 2021.

(51) Int. Cl. G16H 80/00; G16H 10/60; G16H 40/00

(52) U.S. Cl. CPC G16H 80/00; G16H 10/60; G16H 40/00

(57) ABSTRACT

A computer-implemented method for deploying database management tools is disclosed. The method may comprise: receiving user data sets including biological data, previous medical history data, or previous schema data for users; determining one or more groups; receiving a first user data set of a first user; identifying a first group for the first user; generating a plurality of schema plans for the first group; identifying a first schema including a first treatment; determining a schedule; transmitting first electronic content; receiving a signal comprising updated information for the first user; parsing the updated information; and modifying the schema based on the parsed updated information.

### CROSS-REFERENCE TO RELATED APPLICATIONS

This application claims priority to U.S. Provisional Application No. 63/169,364 filed Apr. 1, 2021, the entire disclosure of which is hereby incorporated herein by reference in its entirety.

### TECHNICAL FIELD

Various embodiments of the present disclosure relate generally to care and disease management, and, more particularly, to identifying cohorts and cohort care plans for providing health related services and communication.

### BACKGROUND

Health care management involves many areas of the health care industry including the organization of care providers, institutions, and resources. For a patient, health care management may include providing patient care and patient care plans, as well as facilitating patient communication with care providers.

Managing a health care system, especially in providing patient care, is based on processing large amounts of data and information. The data and information are used by a health care system to determine many things, such as patient diagnosis, patient care plans, costs, follow-up requirements, etc. It is imperative that a health care system manages and utilizes data and information effectively to provide timely and reliable health care services for patients and providers.

Gathering and processing information, however, may be problematic as different health care system use various reporting systems, formats, or methods that create incompatibilities when transferring, sharing and processing health care information. In additional, the amount of data available for processing makes it impossible for care practitioners to consider every piece of information to determine a care plan with the highest chance for success for a particular patient. However, conventional techniques, including the foregoing, fail to provide an effective method for care and disease management.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for care and disease management.

In one aspect, the techniques described herein relate to a computer-implemented method for deploying care management tools, the method including: receiving user data sets for a plurality of users, the user data sets including at least one of biological data, previous medical history data, or previous schema data for the users; determining one or more groups for the plurality of users based on the plurality of data sets; receiving a first user data set of a first user, the first user data set including at least one of the first user's biological data, previous medical history data, or previous schema data; identifying, based on the first user data set, a first group for the first user; generating a plurality of schema plans for the first group; identifying a first schema from the plurality of schemas, based on the first user data set, the first schema including a first treatment from a plurality of treatments; determining a schedule for the first user based on the first schema; transmitting, to the first user based on the schedule, first electronic content comprising the first treatment of the first schema; receiving a signal comprising updated information for the first user; parsing the updated information; and modifying the first schema based on the parsed updated information.

In another aspect, the techniques described herein relate to a system for deploying database management tools, the system comprising: receiving user data sets for a plurality of users, the user data sets including at least one of biological data, previous medical history data, or previous schema data for the users; determining one or more groups for the plurality of users based on the plurality of data sets; receiving a first user data set of a first user, the first user data set including at least one of the first user's biological data, previous medical history data, or previous schema data; identifying, based on the first user data set, a first group for the first user; generating a plurality of schema plans for the first group; identifying a first schema from the plurality of schemas, based on the first user data set, the first schema including a first treatment from a plurality of treatments; determining a schedule for the first user based on the first schema; transmitting, to the first user based on the schedule, first electronic content comprising the first treatment of the first schema; receiving a signal comprising updated information for the first user; parsing the updated information; and modifying the first schema based on the parsed updated information.

In another aspect, the techniques described herein relate to a non-transitory computer-readable medium storing instructions for executing a real-time transaction, the instructions, when executed by one or more processors, causing the one or more processors to perform operations comprising: receiving user data sets for a plurality of users, the user data sets including at least one of biological data, previous medical history data, or previous schema data for the users; determining one or more groups for the plurality of users based on the plurality of data sets; receiving a first user data set of a first user, the first user data set including at least one of the first user's biological data, previous medical history data, or previous schema data; identifying, based on the first user data set, a first group for the first user; generating a plurality of schema plans for the first group; identifying a first schema from the plurality of schemas, based on the first user data set, the first schema including a first treatment from a plurality of treatments; determining a schedule for the first user based on the first schema; transmitting, to the first user based on the schedule, first electronic content comprising the first treatment of the first schema; receiving a signal comprising updated information for the first user; parsing the updated information; and modifying the first schema based on the parsed updated information.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment for care management, according to one or more embodiments.

FIG. 2 depicts an exemplary method for care management, according to one or more embodiments.

FIG. 3 depicts a flowchart of an exemplary method for care management, according to one or more embodiments.

FIGS. 4A-4B depict exemplary embodiments of a graphics user interface (GUI), according to one or more embodiments.

FIG. 5 depicts an exemplary method of using a trained machine learning model to implement the method, according to one or more embodiments.

FIG. 6 depicts an example of a computing device, according to one or more embodiments.

FIG. 7 depicts a flowchart of an exemplary method, according to one or more embodiments.

### DETAILED DESCRIPTION OF EMBODIMENTS

The following embodiments describe systems and methods for facilitating care and disease management. More particularly, the embodiments contemplated in the present disclosure may allow health care management systems to utilize immense amounts of data and information to generate groupings and determine care plans (e.g. “care schemas” or “schemas”). The care plans may be based on the generated groupings providing an efficient method of categorizing a user into a group. A user or group specific care plan may be provided. Thus, aspects of this disclosure provide a technical solution to technical problems associated with data management. By utilizing aspects of the disclosure herein, updates in data information may be analyzed and implemented quickly, seamlessly, and effectively. Communication between a user and a care provider may be instantaneous. Updates regarding efficacy in care procedures or experimental trials are able to be applied to other patients based on the organization of groups/cohorts.

Various aspects of existing health care management systems involve certain drawbacks and deficiencies in providing care schemas to a user. For example, providing care schemas, recommended treatments, and fulfilling outreach requirements may be complicated and inefficient due to various systems, reporting regulations, and an absence of standardization across various demographic markets and health care providers. This affects care providers and their ability to provide health care schemas, as well as users and their experience with receiving treatment and enhanced clinical outcomes.

To address the above-noted problems, the present disclosure describes systems and methods that facilitate an improved health care management system that provides, for example: 1) cohort/group identification; 2) care plan/schema; 3) treatment recommendations; and 4) outreach plans. For example, processing information from a user in combination with information from multiple other users and care providers may generate a health care schema and accompanying information to help a user receive medical treatment.

At a high level, one exemplary embodiment includes a user supplying information related to current symptoms and past medical history and information. This information is analyzed against other information to determine a cohort group to which the user belongs. Based on the cohort, a care plan is selected for the user which includes treatment details tailored specifically for the user. Outreach to the user is scheduled to provide follow-up assessments and to monitor the progress of the user's health.

In this high level exemplary embodiment, artificial intelligence and machine learning may be used for the processing of information to identify groups, cohorts, care schemas, treatments, and outreach. It should be appreciated that particular consideration is made herein to health care management systems and related functions and processes. By training a machine learning model, e.g., via supervised or semi-supervised learning, to learn associations between prior groupings/cohorts based on prior other users, the trained machine learning model may be used to extract prior groups/cohorts in response to the input of first user data. In another exemplary embodiment, a machine learning model may be trained to adjust its biases, weights, and/or layers based on associations between prior schema/plans based on other prior other users. The trained machine learning model may be used to extract schemas/plans in response to the input of the first user data (e.g., a given user's data). Despite this reference to health care management systems, certain disclosed systems and methods may apply equally well to various care management systems.

Systems and methods disclosed herein may be applied to any circumstance where care management includes health care, physical therapy, psychiatric therapy, long-term care, hospice care, where mental or physical health services are provided to a patient, and/or the like. Further, while the user seeking health care services may be referred to as a patient, a user may be referred to herein as a business, a merchant, or a consumer. Furthermore, a user seeking a health care schema need not be a business, a merchant, or a consumer, but may be a financial institution, a government institution, a service provider, or any party seeking health care services.

Embodiments of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” as used herein does not necessarily refer to a different embodiment. It is intended, for example, that claimed subject matter include combinations of exemplary embodiments in whole or in part.

It should be understood that embodiments in this disclosure are exemplary only, and that other embodiments may include various combinations of features from other embodiments, as well as additional or fewer features. For example, while some of the embodiments above pertain to user data (e.g. patient user data), any suitable activity may be used.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially,” “approximately,” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, terms like “user” or “customer” generally encompasses any person or entity that may desire information, resolution of an issue, purchase of a product, or engage in any other type of interaction with a provider. The term “browser extension” may be used interchangeably with other terms like “program,” “electronic application,” or the like, and generally encompasses software that is configured to interact with, modify, override, supplement, or operate in conjunction with other software. As used herein, terms such as “user data” or the like generally encompass patient data, or data pertaining to one or more medical patients. As used herein, a “cohort,” “group,” “population” or the like generally encompasses a plurality of members having definable similarities. As used herein, “plan,” “schema” and the like, generally encompasses care plans, care instructions, or care designs that provide care plans, preventive plans, and/or a detailed or high level general outline or summary of an overall care solution. As used herein, terms such as “real-time transaction” may include monetary transactions or non-monetary transactions such as data information exchange, data packet exchange, or data processing. Real-time transactions may be processed immediately as they occur without any delay for accumulation and result in a transaction record/history that reflects the current status.

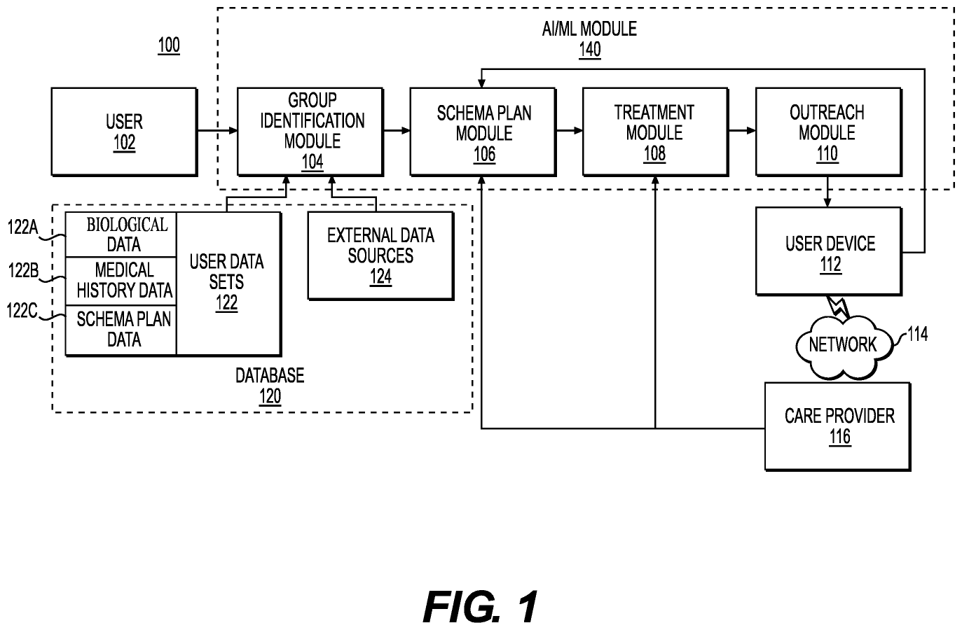

Referring now to the appended drawings, FIG. 1 depicts an environment 100 and how the system relates to a user 102. In some embodiments, the components of environment 100 are associated with a common entity, e.g., a healthcare institution, a healthcare insurer, a population health management company, or the like. In some embodiments, one or more of the components of the environment is associated with a different entity than another. The systems and devices of environment 100 may communicate in any arrangement. As will be discussed herein, systems and/or devices of environment 100 may communicate in order to one or more of generate, train, or use a machine learning model to implement a data fabric system, among other activities.

Although depicted as separate components in FIG. 1, it should be understood that a component or portion of a component in environment 100 may, in some embodiments, be integrated with or incorporated into one or more other components. For example, the AI/ML module 140 may be integrated into one or multiple modules or the like. In another example, user data sets 122 and external data sources 124 may both be integrated a database. In some embodiments, operations or aspects of one or more of the components discussed above may be distributed amongst one or more other components. Any suitable arrangement and/or integration of the various systems and devices of environment 100 may be used.

Included in FIG. 1 is a database 120 that includes user data sets 122 and external data sources 124 and provides information to group identification module 104. The information in user data sets 122 may include biological data 122a, medical history data 122b, schema plan data 122c, or other related data and information. Similarly, external data sources 124 may also include other related data and information. Group identification module 104 also receives relevant data and information specific to user 102 and analyzes this information, in combination with user data sets 122 and external data sources 124, to identify a cohort for user 102.

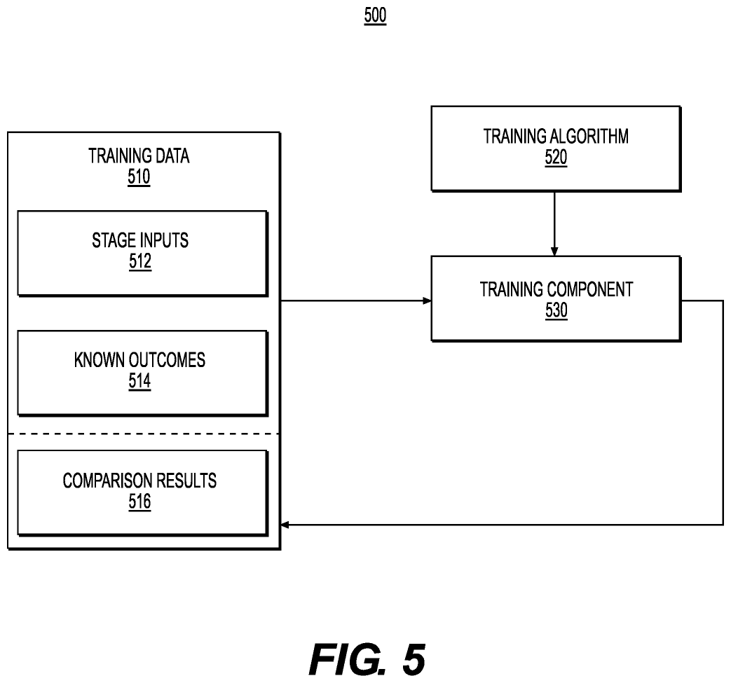

Group identification module 104 may identify and create cohort groupings based on multiple variables such as location, age, disease, virus, injury, health care plan, treatment hospital, or any other relevant demographic or health related category. Group identification module 104 also determines, based on the above mentioned variables and information, which cohort group is to be assigned to user 102. Cohort identification of user 102, or creation of the identified cohorts in general, may be determined using an algorithm or through an AI/ML module 140 by implementing a machine learning model 500 depicted in FIG. 5 and discussed below.

A care plan may be identified using schema plan module 106, based on the identified cohort for user 102. Care plans may be stored in a database 120 and are based on the group cohort. Care plans may include guidelines, and/or timelines for user 102 and describe processes, procedures, and/or overall strategies to improve the health of user 102. Schema plan module 106 may be determined by care providers, health care researchers, or other medical professionals, based on any and all relevant information. Alternatively, schema plan module 106 may utilize AI/ML module 140 by implementing a machine learning model 500 depicted in FIG. 5 and discussed below.

An exemplary embodiment may also include a physician's treatment module 108 that determines, based on the care plan and information about user 102, upcoming treatment for user 102. As the care plan may include general guidance for user 102, the treatment may include more specific details outlining the steps and procedures that are to be taken by user 102. Examples of various physician treatments may include storing of prescription recommendations, dosage amounts, frequency, exercises, procedures, diet, etc. Treatment module 108 may be determined by care providers, health care researchers, or other medical professionals. Alternatively, treatment module 108 may utilize AI/ML module 140 by implementing a machine learning model 500 depicted in FIG. 5 and discussed below.

Another exemplary embodiment may include an outreach module 110. An essential component of providing health care services includes communication with and monitoring of user 102. Outreach module 110 may include any number of communication and follow-up methods including, for example, questionnaires, assessments, surveys, phone calls, text messages, chat, video conferencing, or email. Outreach module 110 may determine when or a frequency to follow-up. Outreach module 110 may also determine the user device 112 to communicate with and provide instructions or facilitate communication with a care provider 116. The outreach module 110 may be determined by care providers, health care researchers, or other medical professionals. Alternatively, outreach module 110 may utilize AI/ML module 140 by implementing a machine learning model 500 depicted in FIG. 5 and discussed below.

In an exemplary embodiment, when outreach module 110 determines that it is time to reach out to user 102 via user device 112, user device 112 receives any of the follow-up communication methods previously described and the user provides a response. The response is communicated over network 114 to care provider 116.

In various embodiments, network 114 may be a wide area network (“WAN”), a local area network (“LAN”), personal area network (“PAN”), or the like. In some embodiments, network 114 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network may obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

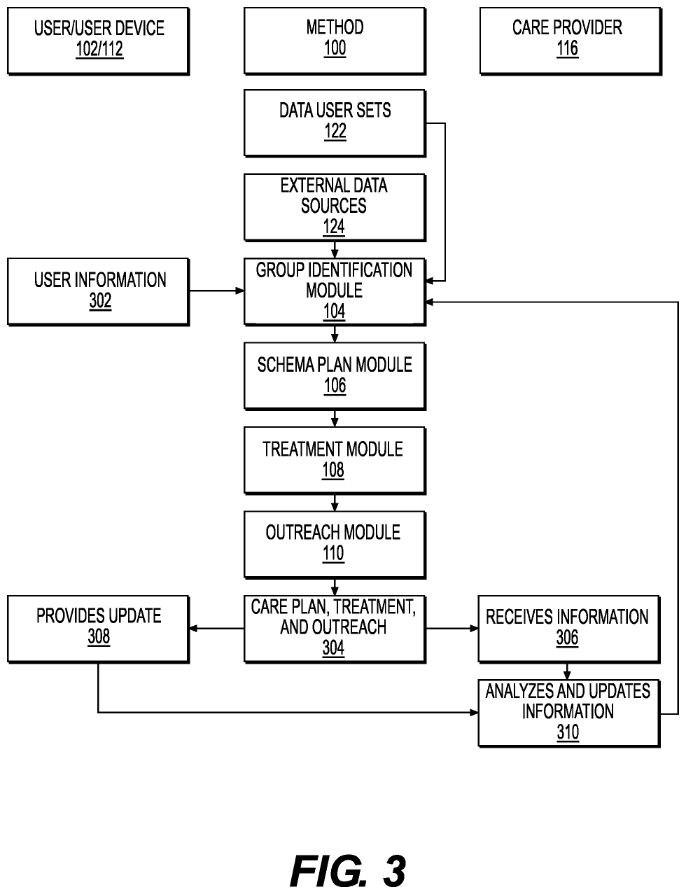

Care provider 116 may analyze the follow-up information, transmitted over network 114, and update the care plan through schema plan module 106 or update the physician treatment plan through treatment module 108. A specific exemplary embodiment related to this environment 100 will be discussed further in FIG. 3.

In the following methods, various acts may be described as performed or executed by a component from FIG. 1. However, it should be understood that in various embodiments, various components of environment 100 discussed above may execute instructions or perform acts including the acts discussed below. An act performed by a device may be considered to be performed by a processor, actuator, or the like associated with that device. Further, it should be understood that in various embodiments, various steps may be added, omitted, and/or rearranged in any suitable manner.

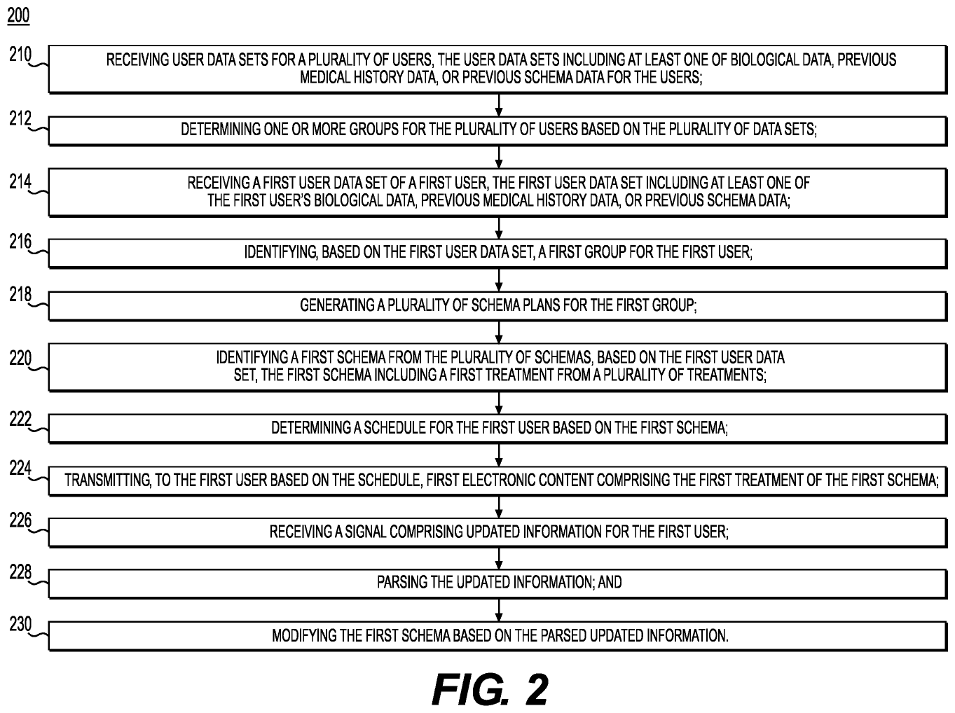

In general, any process or operation discussed in this disclosure that is understood to be computer-implementable, such as the processes illustrated in FIGS. 2, 3, 5 and 7, may be performed by one or more processors of a computer system, such as any of the systems or devices in environment 100 of FIG. 1, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 1. One or more processors of a computer system may be included in a single computing device or distributed among a plurality of computing devices. A memory of the computer system may include the respective memory of each computing device of the plurality of computing devices.

FIG. 2 depicts an exemplary embodiment of flowchart 200, according to one or more embodiments. In an exemplary embodiment, the method includes techniques relating to a computer-implemented method for deploying care management tools. At step 210, an exemplary method includes receiving, by a processor, user data sets that include biological data, previous medical history data, or previous schema data for the users. Biological data may include age, height, weight, cholesterol levels, heart rate, etc. Previous medical history data may include allergies, surgeries, hospitalizations, or any history related to cancer, diabetes, heart disease, or any other applicable health related condition. Previous care plan/schema data may include treatment care plans, weight loss goals, exercise plans, meal or dietary schemas, sobriety plans and/or the like. At step 212, an exemplary embodiment also includes determining one or more groups/cohorts for each of the users based on the data sets. For example, a group of users having diabetes may be one cohort group. Another cohort group may be 39 year old males that live in Salt Lake City, Utah. Another cohort group may be dependents on insurance plan ABC. Cohort groups may be based on a single factor or multiple factors and users may be included in multiple groups/cohorts. At step 214, an exemplary embodiment may further include receiving a first user data set of a first user that includes the first user's biological data, previous medical history data, or previous schema data. For example, biological data for the first user include information such as 28 years old, female, living in Kailua, Hi. An example of medical history may include a melanoma removal procedure in 2012 and a kidney infection in 2017. Exemplary previous schema data may include dietary restrictions for dairy products.

At step 216, an exemplary embodiment may include identifying a first group/cohort for the first user based on the first user data set. As an example, a group for the user may be “sunburnt, high risk skin cancer patient” and may be determined based on the data of the first user, that the first user is 28 years old, had a melanoma removal procedure performed, lives in a state with a high sun index, and is sun burnt. At step 218, an exemplary embodiment may include generating a plurality of schema/care plans for the group. In the exemplary embodiment, the “sunburnt, high risk skin cancer patient” group may include a variety of schema/care plans. For example, schema plans may include no direct sunlight, minimal direct sunlight, and approved for direct sunlight.

At step 220, an exemplary embodiment may include identifying a first schema for the user based on the user data set, along with a treatment determined. For example, a first schema may consider that the first user is 28 years old, had a melanoma removal procedure performed, lives in a state with a high sun index, and is sun burnt, and identify a schema plan for the patient that includes no direct sunlight. The treatment, as directed by a physician, may include applying aloe vera skin cream, and for the future, no direct sunlight longer than 15 minutes without sunscreen and full covering for direct sunlight lasting longer than 15 minutes. In one exemplary embodiment, the schema and treatment are determined by medical professionals. Alternatively, the schemas and treatments can be determined by AI/ML which will be discussed below.

At step 222, an exemplary embodiment may also include determining a schedule for the first user based on the first schema. The schedule may include how often to apply aloe vera. The schedule may include when to have a follow-up appointment. The schedule may include a suggested day for seeing a dermatologist to track and monitor skin tags or moles, or to scan for other signs of melanoma. In one exemplary embodiment, the schedule may be determined by medical professionals, or alternatively, determined by AI/ML which will be discussed below.

At step 224, an exemplary embodiment may include transmitting electronic content, including the treatment and schema for the first user, to the first user. The transmission may be based on the schedule and may include network communication from one device to a user device. At step 226, an exemplary embodiment may include receiving a signal that includes updated information for the first user. In one case, the user may have provided communication from the user device over an internet connection to provide updated information that includes that the sunburn has gone away. At step 228, the updated information may be parsed and, at step 230, the schema may be determined based on the parsed information. In an example embodiment, the updated information that the sunburn has gone away is parsed to determine that the first schema can be modified for “no direct sunlight” to a schema such as “minimal sunlight.”

In another exemplary embodiment, the first electronic content includes email, messaging, video, surveys, or information. In another embodiment, the techniques further include transmitting the updated information to a care provider and connecting the care provider with the first user for real-time communication. Exemplary embodiments may include connecting a physician with the first user via video chat or telephone.

In another exemplary embodiment, the techniques related to the method further include receiving feedback signals from a first user device, determining a schema success rate based on the feedback signals, and modifying the plurality of schemas for the first group, based on the feedback signals. An exemplary embodiment may include the first user providing feedback that use of aloe vera significantly decreased the healing time for the sunburn. This information may be found to be consistent among other users that also suffered from burns and used aloe vera. The exemplary embodiment may modify all schemas relating to sunburn and include aloe vera treatment in all of the schemas relating to sunburn.

In another exemplary embodiment, the techniques related to the method further include receiving feedback signals from a first user device, determining a care plan success rate based on the feedback signals, and modifying the care plan for the first user, based on the feedback signals. An exemplary embodiment may include the first user providing feedback that use of aloe vera significantly decreased the healing time for the sunburn and determine a success rate when using aloe vera to treat sunburn. The schema/care plan for the user may be modified to allow for minimal direct sunlight due to already having a past melanoma removal surgery.

In another exemplary embodiment, the techniques related to the method further include identifying the first cohort/group for the first user based on a group/cohort machine learning model output. In another exemplary embodiment, the techniques related to the method further include the care plan being identified based on an output of a care plan machine learning model output.

FIG. 3 depicts a flowchart 300 of an exemplary method. Specifically, FIG. 3 includes flowchart 300 outlining the interactions of user/user device 102/112 with care provider 116. As an example illustration, FIG. 3 will be described in relation to a specific embodiment, the COVID-19 virus, for exemplary purposes.

As an exemplary embodiment, database 120 (not shown) provides user data sets 122 and external data sources 124 to group identification module 104. The user data sets may include specific information for the user relating to biological data 122a (not shown in FIG. 3), such as age, weight, and blood pressure, medical history data 122b (not shown in FIG. 3) such as asthma, respiratory issues, etc., and schema plan data 122c (not shown in FIG. 3) such as inhaler use, vaccinations, or other related data and information.

As an exemplary embodiment, user 102 provides user information 302 to group identification module 104. In this exemplary embodiment, user information 302 may include that user 102 has flu like symptoms and has lost their sense of taste. Group identification module 104 is able to process this information, in combination with user data sets 122 and external data sources 124 to identify a cohort grouping of COVID-19 patients who have similar symptoms to user 102.

Cohort identification or grouping may employ a number of methods to accurately determine the cohort grouping for the user. For example, group identification module 104 may utilize a threshold or confidence value system where a quantitative score for a grouping is determined (based on the information of the user) and the score exceeds a threshold or is within an acceptable confidence interval. Additionally, processing the available information to determine a cohort grouping may be done either by care professionals or using AI/ML. In this exemplary embodiment, group identification module 104 processes the information to determine that the user belongs to a COVID-19 candidate group.

A group or cohort can be dynamic. New users may be added to an existing cohort and existing users may be removed from an existing cohort/group. Additionally, a group or cohort may be static. For example, a static cohort/group may not be modified once the cohort/group is established.

In this exemplary case, schema plan module 106 will use the cohort/group of user 102, user information 302 and user data sets 122 to determine that the user's schema/care plan would also consider the user's history of having asthma. A schema or plan may include an overarching idea or general strategy for medical treatment.

As an example, schema plan module 106 may factor in the patient's asthma to determine that the schema/care plan for the user should be escalated in comparison to a care plan for a non-asthmatic patient. In this instance, generation of the schema/care plan for user 102 may be based on information processed from external data sources 124 and other user data sets. That information may provide insight based on those in similar situations as user 102 requiring increased levels of care and attention due to the a higher likelihood of increased long-term lung damage and higher fatality rates. In this exemplary embodiment, the patient may have a schema plan that is a “high risk” schema plan relating to the COVID-19 candidate group. Alternatively, there may also be a “low risk” schema plan relating to the COVID-19 candidate group.

Based on the schema/care plan for user 102, treatment module 108 may determine a treatment plan for user 102 based on the user's information and how it compares or tracks based on other patients having similar biological data and medical history.

In one exemplary embodiment, a treatment plan may be automatically generated based on selecting “components” from a library of available components. Examples of components can include available treatments, drugs, educational materials, at-home exercises, etc. The components may be selected based on past treatment plans or may be generated using an AI/ML model. In addition, a packaged treatment plan may further be modified based on specific data of the user/patient. The modification of the packaged treatment plan may be implemented by an overseeing care physician, generated using an algorithm, or determined by an AI/ML model.

For example, treatment for user 102 as an asthmatic patient might include immediate quarantine, taking a COVID-19 test and physician notification for medication to treat symptoms. In comparison, a treatment recommendation for a non-asthmatic patient may simply include providing COVID-19 information material and medication to treat symptoms, and a quarantine recommendation for 10 days. Multiple treatment plans may exist for a single group and for a single schema. In treatment module 108, treatments may be determined by care specialists and professionals, algorithms, or through AI/ML module 140 by implementing a machine learning model 500 depicted in FIG. 5 and discussed below.

In this exemplary embodiment, outreach module 110 will consider and process the information to create an outreach plan that can describe a type and frequency of follow-up. In this example, with the increased risk of the asthmatic user, the outreach plan may include hourly check-ins with the user to ensure frequent monitoring. In comparison, a non-asthmatic outreach plan determined by outreach module 110 may require a check-in after 5 days of being diagnosed, simply to monitor progress.

In an exemplary embodiment, the care plan, treatment, and any outreach 304 is provided to user 102. Care provider 116 also receives information 306 of care plan, treatment, and any outreach 304. Based on the information, care provider 116 analyzes and updates information 310, to be fed back into the group identification module 104. According to some aspects of the disclosure, the additional information may generate a different cohort grouping necessitating updates to the care plan, treatment, and outreach. In another exemplary embodiment, user 102 provides an update 308 which is communicated to care provider 116. For example, new information may include an indication that the user is having difficulty breathing. The care provider analyzes and updates information 310 and feeds the analyzed and updated information into group identification module 104 to determine if any changes to the cohort grouping are required. Any changes to the cohort/group identification may result in necessary changes to the care/schema plan, treatment, and/or outreach as described above.

In this example, according to some aspects, the new information indicating difficulty breathing may result in the cohort/group for the user being changed to a COVID-19 shortness of breath group and creating an updated care plan that includes an escalation of observation and respiratory care. The treatment module 108 may be updated to include patient referral to inpatient care and urgent notification of family members. In an exemplary embodiment, this information may be provided as an update to the user (and in this case, the user's family).

As a continuation of this exemplary embodiment, the user may provide an update indicating that, after being hospitalized on a ventilator for a week, they no longer have a shortness of breath and are feeling well indicating discharge planning should be executed. Care provider 116 will analyze and update the information and provide the update to group identification module 104. The updated information may result in a new cohort group for the user, that of COVID-19 recovered patients, which results in a care plan adjustment to day-to-day monitoring and a treatment plan to rest and avoid intense cardio exercise. The outreach module 110 may require an update in a few days and the care plan, treatment, and outreach 304 may be provided to user 102 through user device 112 and to care provider 116 who analyzes and updates the information 310.

In these exemplary embodiments, updates in information may be analyzed and implemented quickly, seamlessly, and effectively. Communication between user 102 and care provider 116 may be instantaneous or near instantaneous (e.g., within 5 minutes of receiving and/or outputting an update). Updates regarding efficacy in procedures and trials are able to be factored and applied to other patients by organization of grouping cohorts and other informational data. These benefits may be faster realized by utilizing AI/ML module 140 and implementing a machine learning model 500 to process and analyze various types of data and information. Additionally, instant and seamless communication to user 102 via user device 112 also accounts for the benefits provided by the process disclosed in flowchart 200.

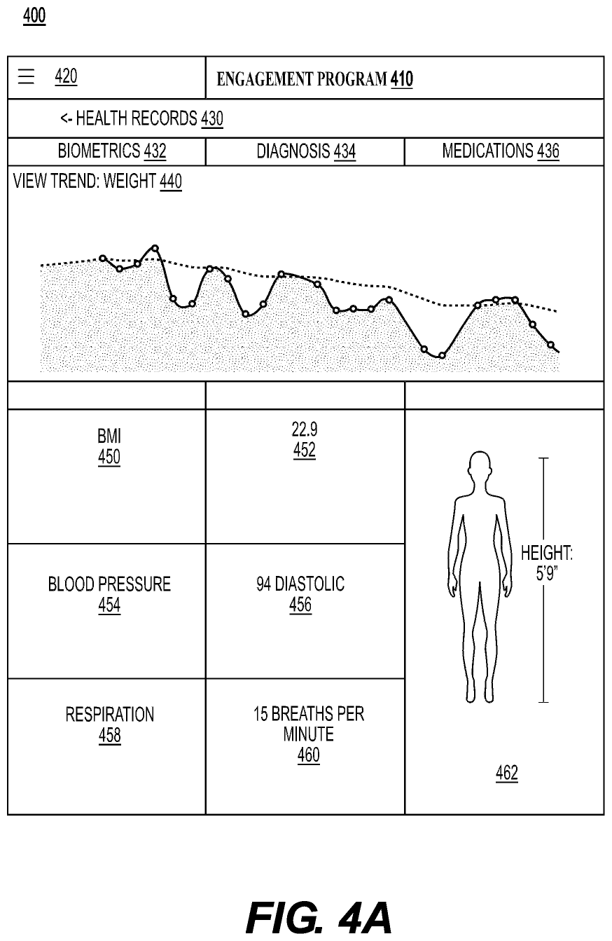

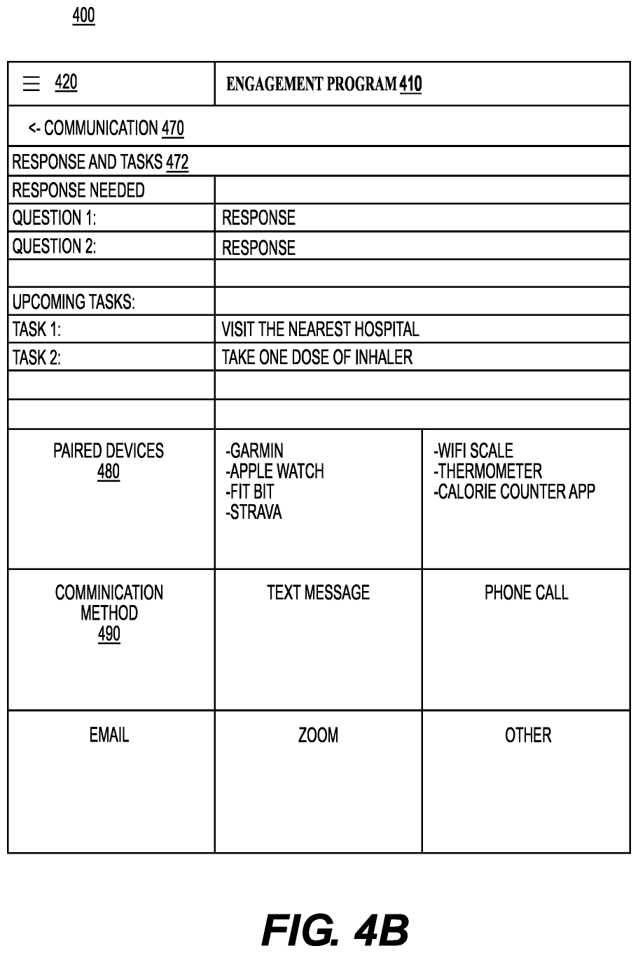

FIGS. 4a and 4b describe an exemplary embodiment of a graphical user interface 400 that may be configured to enable a health care provider or user to access and/or interact with other systems in environment 100. For example, graphical user interface 400 may be implemented on user device 112 as a computer system such as, for example, a desktop computer, a mobile device, a tablet, etc. In some embodiments, graphical user interface 400 may be associated or implemented by user device 112 that may include one or more electronic application(s), e.g., a program, plugin, browser extension, etc., installed on a memory of the user device. In some embodiments, the electronic application(s) may be associated with one or more of the other components in environment 100. For example, the electronic application(s) may include one or more of system control software, system monitoring software, software development tools, etc.

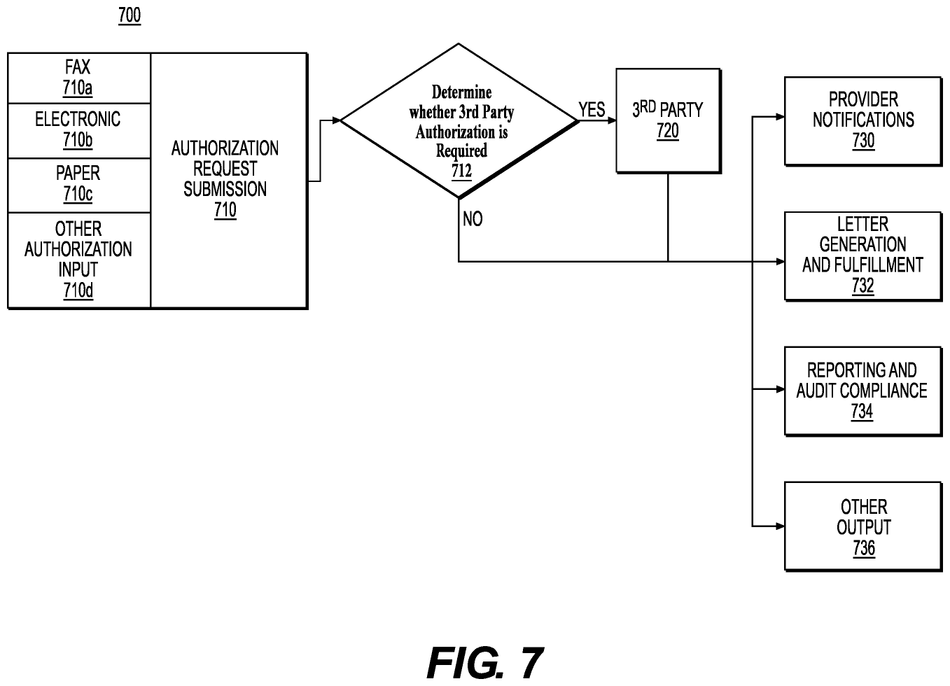

User device 112 associated with graphical user interface 400 may include a server system, an electronic medical data system, computer-readable memory such as a hard drive, flash drive, disk, etc. In some embodiments, the user device associated with graphical user interface 400 includes and/or interacts with an application programming interface for exchanging data to other systems, e.g., one or more of the other components of the environment. The user device may include and/or act as a repository or source for user data/patient related data. For example, data from care provider software may be utilized by others, such as 3^{rd} parties, as discussed in more detail below in FIG. 7.

As an exemplary embodiment, FIG. 4a depicts a graphical user interface 400 of a health records tab 430. The graphical user interface 400 portrays an exemplary embodiment of an engagement program 410 with a hamburger menu 420 allowing user 102 to navigate to other tabs of engagement program 410.

The exemplary embodiment of health records tab 430 includes three display tabs, biometrics 432, diagnosis tab 434, and medications tab 436. Other tabs and relevant information may also be included such as history tab displaying recent and past medical procedures, or an events tab displaying any upcoming activities or events, such as doctor appointments or physical therapy sessions. In this exemplary embodiment, the biometrics tab is being viewed and displays a view trend: weight 440 and a graphical representation tracking the user's weight. In another embodiment, the view trend may be caloric intake, active calories, user input self-assessment health ratings, or other graphical representations. Diagnosis tab 434 and medications tab 436 may also populate corresponding information relevant to the health records of user 102.

In an exemplary embodiment, FIG. 4a includes body mass index or BMI 450 and a BMI value 452, blood pressure 454 and a blood pressure value 456, and a respiration measurement 458 and a respiration value 460. Body mass index is a health ratio based on height and weight. Blood pressure is the force that moves blood through the circulatory system. A respiration measurement breathes taken per minute can assist with determining respiratory issues. A graphical depiction of the user and a height of user 462 may also be displayed as shown in FIG. 4a. Other informational headings and data may also be included. The information may be transmitted over network 114, or input by user 102 or care provider 116.

As an exemplary embodiment, FIG. 4b depicts graphical user interface 400 of a communication tab 470 that facilitates communication between user 102 and care provider 116. Communication tab 470 may apply the communication referenced above in FIG. 1 between user device 112 over network 114 with care provider 116. Communication tab 470 may also relate to communication in FIG. 3 between user device 112 and care provider 116 such as when user 102 transmits a care plan, treatment, and outreach 304, provides an update 308, and analyzes and updates information 310. Graphical user interface 400 portrays an exemplary embodiment of engagement program 410 with hamburger menu 420 allowing a user 102 to navigate to other tabs of engagement program 410, such as health records tab 430.

In an exemplary embodiment, communication tab 470 includes a section for response and tasks 472. Details in this exemplary embodiment include responses needed followed by questions and a section for user 102 to provide corresponding responses. To continue with an exemplary embodiment, a question may be, “How difficult is it for you to breath?” The user 102 could respond in the respond section with, “I am having difficulty breathing.” This may generate upcoming tasks such as a task 1 of “cough and deep breathing exercises” and a task 2 of “take one dose of the inhaler.”

In another exemplary embodiment, paired devices 480 may list any devices or applications that are connected to engagement program 410. These connected devices or applications may include Garmin, apple watch, fit bit, Strava, Wi-Fi scale, thermometer, calorie counter, etc. These connected devices or applications may provide information and data that is able to be utilized by engagement program 410 and relayed to care provider 116. Additionally, the information may be used to update user information 302 which will assist to ensure that cohort information, care plans, and treatments are utilizing as much information as possible to provide care recommendations based on a comprehensive review of information currently available. In this exemplary embodiment, communication methods 490 provide an easy one-click method to begin a text message, phone call, email, zoon, or other form of communication.

FIG. 5 depicts a flow diagram for training machine learning model 500. In some embodiments, a system or device may include instructions for generating machine learning model 500, training data 510 and ground truth, and/or instructions for training the machine learning model. A resulting trained-machine learning model may then be provided to AI/ML module 140.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of machine learning model 500 may operate on an input linearly, in parallel, via a network (e.g., a neural network), or via any suitable configuration.

The execution of machine learning model 500 may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

Generally, machine learning model 500 includes a set of variables, e.g., nodes, neurons, filters, etc., that are tuned, e.g., weighted or biased, to different values via the application of training data. In supervised learning, e.g., where a ground truth is known for training data 510 provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of training data 510 may be withheld during training and/or used to validate machine learning model 500, e.g., compare the output of the trained model with the ground truth for that portion of the training data to evaluate an accuracy of the trained model. The training of machine learning model 500 may be configured to cause machine learning model 500 to learn associations between prior groupings/cohorts based on prior other users. The machine learning model may be usable to extract prior groups/cohorts in response to the input of first user data. In another exemplary embodiment, training a machine learning model to learn associations between prior schema/plans based on other prior other users may be usable to extract schemas/plans in response to the input of the first user data. In general, machine learning model 500 is configured to determine an output data in response to the input data based on the learned associations.

In various embodiments, the variables of machine learning model 500 may be interrelated in any suitable arrangement in order to generate the output. For example, in some embodiments, machine learning model 500 may include image-processing architecture that is configured to identify, isolate, and/or extract features, geometry, and or structure in one or more of the medical imaging data and/or the non-optical in vivo image data. In some instances, different samples of training data and/or input data may not be independent. Thus, in some embodiments, machine learning model 500 may be configured to account for and/or determine relationships between multiple samples.

In one embodiment, machine learning model 500 implements a targeted cohort grouping. One or more implementations disclosed herein may be applied by using machine learning model 500. A machine learning model as disclosed herein may be trained using environment 100 of FIG. 1, flowchart 200 of FIG. 2, flowchart 300 of FIG. 3, and/or flowchart 700 of FIG. 7. As shown in the flow diagram of FIG. 5, training data 510 may include one or more of stage inputs 512 and known outcomes 514 related to machine learning model 500 to be trained. Stage inputs 512 may be from any applicable source including a component or set shown in FIGS. 1, 2, 3, and/or 7. The known outcomes 514 may be included for machine learning models generated based on supervised or semi-supervised training. An unsupervised machine learning model might not be trained using known outcomes 514. Known outcomes 514 may include known or desired outputs for future inputs similar to or in the same category as stage inputs 512 that do not have corresponding known outputs.

Training data 510 and a training algorithm 520 may be provided to a training component 530 that may apply training data 510 to training algorithm 520 to generate AI/ML module 140. According to an implementation, training component 530 may be provided comparison results 516 that compare a previous output of machine learning model 500 to apply the previous result to re-train machine learning model 500. Comparison results 516 may be used by training component 530 to update machine learning model 500. Training algorithm 520 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like. The output of machine learning model 500 may be a trained machine learning model.

According to some aspects of the disclosure, the relevant data may be extracted from the modified user data using a trained machine learning model. According to this aspect of the disclosure, the trained machine learning model may be trained to extract relevant data from the modified user data based on (i) training a machine learning model to learn associations between prior groupings/cohorts based on prior other users to extract prior groups/cohorts in response to the input of first user data, and (ii) training a machine learning model to learn associations between prior schema/plans based on other prior other users to extract schemas/plans in response to the input of the first user data.

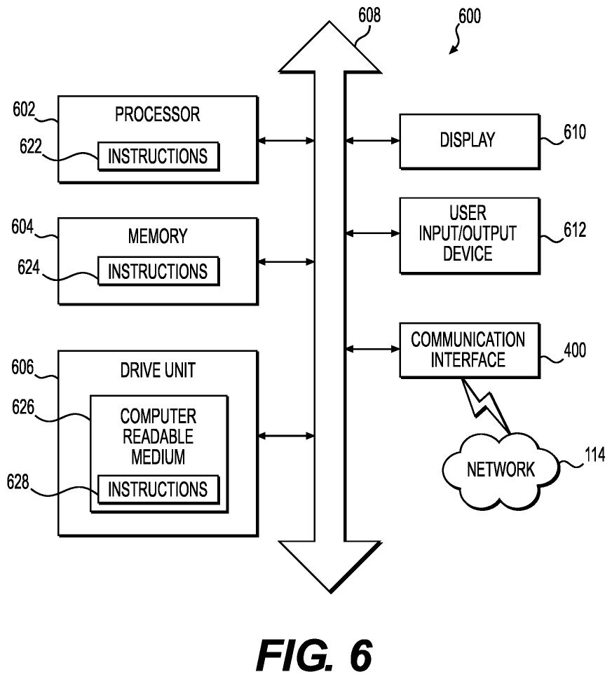

FIG. 6 illustrates a computer system 600. In general, FIG. 6 may represent any process or operation discussed in this disclosure that is understood to be computer-implementable, such as the processes illustrated in FIGS. 1, 2, 3, 5 and 7, and may be performed by one or more processors of computer system 600, such as any of the systems or devices in computer system 600 of FIG. 6. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory 604 of the computer system 600. A processor 602 may be a central processing unit (CPU), a graphics processing unit (GPU), or any suitable types of processing unit.

Computer system 600, such as a system or device implementing a process or operation in the examples above, may include one or more computing devices, such as one or more of the systems or devices in FIG. 6. One or more processors of a computer system may be included in a single computing device or distributed among a plurality of computing devices. A memory of the computer system may include the respective memory of each computing device of the plurality of computing devices.

Unless specifically stated otherwise, it is appreciated that throughout the specification discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer,” a “computing machine,” a “computing platform,” a “computing device,” or a “server” may include one or more processors.

Computer system 600 may include a set of instructions that may be executed to cause computer system 600 to perform any one or more of the methods or computer based functions disclosed herein. Computer system 600 may operate as a standalone device or may be connected, e.g., using network 114, to other computer systems or peripheral devices.

In a networked deployment, computer system 600 may operate in the capacity of a server or as a client user computer in a server-client user network environment, or as a peer computer system in a peer-to-peer (or distributed) network environment. Computer system 600 may also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, computer system 600 may be implemented using electronic devices that provide voice, video, or data communication. Further, while a computer system 600 is illustrated as a single computer system, the term “system” may also be taken to include any collection of systems or sub-systems that individually or jointly execute a set, or multiple sets, of instructions to perform one or more computer functions.

As illustrated in FIG. 6, computer system 600 may include processor 602, e.g., a central processing unit (CPU), a graphics processing unit (GPU), or both. The processor 602 may be a component in a variety of systems. For example, processor 602 may be part of a standard personal computer or a workstation. Processor 602 may be one or more general processors, digital signal processors, application specific integrated circuits, field programmable gate arrays, servers, networks, digital circuits, analog circuits, combinations thereof, or other now known or later developed devices for analyzing and processing data. Processor 602 may implement a software program, such as code generated manually (i.e., programmed) and/or include by instructions 622.

Computer system 600 may include a memory 604 that may communicate via a bus 608. Memory 604 may be a main memory, a static memory, or a dynamic memory. Memory 604 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, memory 604 includes a cache or random-access memory for processor 602. In alternative implementations, the memory 604 is separate from processor 602, such as a cache memory of a processor, the system memory, or other memory. Memory 604 may be an external storage device or database for storing data. Examples include a hard drive, compact disc (“CD”), digital video disc (“DVD”), memory card, memory stick, floppy disc, universal serial bus (“USB”) memory device, or any other device operative to store data. Memory 604 is operable to store instructions 624 executable by processor 602. The functions, acts or tasks illustrated in the figures or described herein may be performed by the processor 602 executing the instructions stored in memory 604. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel payment and the like.

As shown, computer system 600 may further include a display 610, such as a liquid crystal display (LCD), an organic light emitting diode (OLED), a flat panel display, a solid-state display, a cathode ray tube (CRT), a projector, a printer or other now known or later developed display device for outputting determined information. Display 610 may act as an interface for the user to see the functioning of processor 602, or specifically as an interface with the software stored in memory 604 or in drive unit 606.

Additionally or alternatively, computer system 600 may include an input device 612 configured to allow a user to interact with any of the components of computer system 600. Input device 612 may be a number pad, a keyboard, or a cursor control device, such as a mouse, or a joystick, touch screen display, remote control, or any other device operative to interact with computer system 600.

Computer system 600 may also or alternatively include a drive unit 606 that is either disk or optical. Drive unit 606 may include a computer-readable medium 626 in which one or more sets of instructions 628, e.g., software, may be embedded. Further, instructions 628 may embody one or more of the methods or logic as described herein. Instructions 628 may reside completely or partially within memory 604 and/or within processor 602 during execution by computer system 600. Memory 604 and processor 602 also may include computer-readable media as discussed above.

In some systems, a computer-readable medium 626 includes instructions 628 or receives and executes instructions 628 responsive to a propagated signal so that a device connected to network 114 may communicate voice, video, audio, images, or any other data over network 114. Further, instructions 628 may be transmitted or received over network 114 via graphical user interface 400, and/or using bus 608. Graphical user interface 400 may be a part of processor 602 or may be a separate component. Graphical user interface 400 may be created in software or may be a physical connection in hardware. Graphical user interface 400 may be configured to connect with network 114, external media, display 610, or any other components in computer system 600, or combinations thereof. The connection with network 114 may be a physical connection, such as a wired Ethernet connection or may be established wirelessly as discussed below. Likewise, the additional connections with other components of the computer system 600 may be physical connections or may be established wirelessly. Network 114 may alternatively be directly connected to bus 608.

While the computer-readable medium 626 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 626 may be non-transitory, and may be tangible.

The computer-readable medium 626 may include a solid-state memory such as a memory card or other package that houses one or more non-volatile read-only memories. The computer-readable medium 626 may be a random-access memory or other volatile re-writable memory. Additionally or alternatively, computer-readable medium 626 may include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, may be constructed to implement one or more of the methods described herein. Applications that may include the apparatus and systems of various implementations may broadly include a variety of electronic and computer systems. One or more implementations described herein may implement functions using two or more specific interconnected hardware modules or devices with related control and data signals that may be communicated between and through the modules, or as portions of an application-specific integrated circuit. Accordingly, the present system encompasses software, firmware, and hardware implementations.

Computer system 600 may be connected to network 114 or multiple networks. Network 114 may define one or more networks including wired or wireless networks. The wireless network may be a cellular telephone network, an 802.11, 802.16, 802.20, or WiMAX network. Further, such networks may include a public network, such as the Internet, a private network, such as an intranet, or combinations thereof, and may utilize a variety of networking protocols now available or later developed including, but not limited to TCP/IP based networking protocols. Network 114 may include wide area networks (WAN), such as the Internet, local area networks (LAN), campus area networks, metropolitan area networks, a direct connection such as through a Universal Serial Bus (USB) port, or any other networks that may allow for data communication. Network 114 may be configured to couple one computing device to another computing device to enable communication of data between the devices. Network 114 may generally be enabled to employ any form of machine-readable media for communicating information from one device to another. Network 114 may include communication methods by which information may travel between computing devices. Network 114 may be divided into sub-networks. The sub-networks may allow access to all of the other components connected thereto or the sub-networks may restrict access between the components. Network 114 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations may include distributed processing, component/object distributed processing, and parallel payment. Alternatively, virtual computer system processing may be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP, etc.) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

FIG. 7 includes a flowchart 700 of an exemplary embodiment that relates a method for care and disease management to 3^{rd} parties. The generation of a care plan and treatments may require authorization from insurers or other care providers, such as specialists. As an exemplary embodiment, FIG. 7 depicts an exemplary embodiment that includes interactions between care provider 116 and a health care insurance provider.

After a care plan/schema and treatment are determined, for example as described above at in FIG. 2, an authorization request submission 710 may be required prior to implementing a care plan and the corresponding care treatment. Authorization request submission 710 may be received via fax 710a, electronic 710b, paper 710c such as any signed or written documents, or any other authorization input 710d. According to an implementation, paper 710c authorization requests may be received via fax 710a, may be scanned and submitted as electronic 710b submissions, or the like. After receiving authorization request submission 710, group identification module 104 (not pictured) may determine whether 3^{rd} party authorization is required 712. In one exemplary embodiment, 3^{rd} party authorization is not required. These cases may be those that are trivial, routine, or inexpensive. In these cases, group identification module 104 may designate authorization request submission 710 as belonging to a “3^{rd} party authorization is not required” group. In the event that 3^{rd} party authorization is not required, flowchart 700 includes transmitting provider notifications 730, letter generation and fulfillment 732, reporting and audit compliance 734, and/or any other output 736.

In another embodiment, 3^{rd} party authorization is determined by group identification module 104 (not pictured) to be required. These cases may include those that are complicated, unique, and/or expensive. In these cases, group identification module 104 may designate authorization request submission 710 as belonging to a “3^{rd} party authorization is required” group. In the event that 3^{rd} party authorization is required, the proposed care plan and treatment may be analyzed and assessed by a 3^{rd} party 720. The 3^{rd} party may perform their own method for determining whether to authorize the care plan and treatment. After 3^{rd} party 720 analyzes the care plan and treatment, the exemplary embodiment includes transmitting provider notifications 730, letter generation and fulfillment 732, reporting and audit compliance 734, and/or any other output 736. It should be appreciated that requiring 3^{rd} party authorization may be iterative. For example, after a 3^{rd} party provides authorization, approval may be required by one or more other parties. According to this example, the number or type of iterations and/or the one or more other parties may be determined based on the type of priority authorization, the cost of the services and/or treatment associated with the prior authorization, or based on patient history.

It should be appreciated that in the above description of exemplary embodiments, various features of the embodiments are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that a claimed embodiment requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment.

Furthermore, while some embodiments described herein include some but not other features included in other embodiments, combinations of features of different embodiments are meant to be within the scope of the present disclosure, and form different embodiments, as would be understood by those skilled in the art. For example, in the following claims, any of the claimed embodiments may be used in any combination.

Furthermore, some of the embodiments are described herein as a method or combination of elements of a method that may be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the function.

In the description provided herein, numerous specific details are set forth. However, it is understood that embodiments of the present disclosure may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. This simply means that a path exists between an output of A and an input of B, which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the present disclosure, those skilled in the art will recognize that other and further modifications may be made thereto without departing from the spirit of the present disclosure, and it is intended to claim all such changes and modifications as falling within the scope of the present disclosure. For example, any formulas given above are merely representative of procedures that may be used. Functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

What is claimed is:

1. A computer-implemented method for deploying database management tools, the method comprising:
receiving user data sets for a plurality of users, the user data sets including at least one of biological data, previous medical history data, or previous schema data for the users;
determining one or more groups for the plurality of users based on the plurality of data sets;
receiving a first user data set of a first user, the first user data set including at least one of the first user's biological data, previous medical history data, or previous schema data;
identifying, based on the first user data set, a first group for the first user;
generating a plurality of schema plans for the first group;
identifying a first schema from the plurality of schemas, based on the first user data set, the first schema including a first treatment from a plurality of treatments;
determining a schedule for the first user based on the first schema;
transmitting, to the first user based on the schedule, first electronic content comprising the first treatment of the first schema;
receiving a signal comprising updated information for the first user;
parsing the updated information; and
modifying the first schema based on the parsed updated information.

2. The computer-implemented method of claim 1, wherein the first electronic content includes email, messaging, video, surveys, information, or combinations thereof.

3. The computer-implemented method of claim 1, further comprising:
transmitting the updated information to a provider; and
connecting the provider with the first user for real-time communication.

4. The computer-implemented method of claim 1, further comprising:
receiving feedback signals from a first user device;
determining a schema success rate based on the feedback signals; and
modifying the plurality of schemas for the first group, based on the feedback signals.

5. The computer-implemented method of claim 1, further comprising:
receiving feedback signals from a first user device;
determining a schema success rate based on the feedback signals; and
modifying the first schema for the first user, based on the feedback signals.

6. The computer-implemented method of claim 1, wherein identifying the first group for the first user is based on a group machine learning model output.

7. The computer-implemented method of claim 1, wherein the schema is identified based on an output of a schema machine learning model output.

8. A system for deploying database management tools, the system comprising:
at least one memory storing instructions; and
at least one processor executing the instructions to perform a process, the processor configured to: receive user data sets for a plurality of users, the user data sets including at least one of biological data, previous medical history data, or previous schema data for the users;
determine one or more groups for the plurality of users based on the plurality of data sets;
receive a first user data set of a first user, the first user data set including at least one of the first user's biological data, previous medical history data, or previous schema data;
identify, based on the first user data set, a first group for the first user;
generate a plurality of schema plans for the first group;
identify a first schema from the plurality of schemas, based on the first user data set, the first schema including a first treatment from a plurality of treatments;
determine a schedule for the first user based on the first schema;
transmit, to the first user based on the schedule, first electronic content comprising the first treatment of the first schema;
receive a signal comprising updated information for the first user;
parse the updated information; and
modify the first schema based on the parsed updated information.

9. The system of claim 8, wherein the first electronic content includes email, messaging, video, surveys, information, or combinations thereof.

10. The system of claim 8, wherein the processor is further configured to:
transmit the updated information to a provider; and
connect the provider with the first user for real-time communication.

11. The system of claim 8, wherein the processor is further configured to:
receive feedback signals from a first user device;
determine a schema success rate based on the feedback signals; and
modify the plurality of schemas for the first group, based on the feedback signals.

12. The system of claim 8, wherein the processor is further configured to:
receive feedback signals from a first user device;
determine a schema success rate based on the feedback signals; and
modify the first schema for the first user, based on the feedback signals.

13. The system of claim 8, wherein identifying the first group for the first user is based on a group machine learning model output and wherein the schema is identified based on an output of a schema machine learning model output.

14. The system of claim 8, wherein the processor is further configured to:
generate an authorization request submission based on the first schema;
transmit the authorization request to a third party;
receive an authorization request approval from the third party; and
transmit the first user content further based on the authorization request approval.

15. A non-transitory computer-readable medium storing instructions for executing a real-time transaction, the instructions, when executed by one or more processors, causing the one or more processors to perform operations comprising:
receiving user data sets for a plurality of users, the user data sets including at least one of biological data, previous medical history data, or previous schema data for the users;
determining one or more groups for the plurality of users based on the plurality of data sets;
receiving a first user data set of a first user, the first user data set including at least one of the first user's biological data, previous medical history data, or previous schema data;
identifying, based on the first user data set, a first group for the first user;
generating a plurality of schema plans for the first group;
identifying a first schema from the plurality of schemas, based on the first user data set, the first schema including a first treatment from a plurality of treatments;
determining a schedule for the first user based on the first schema;
transmitting, to the first user based on the schedule, first electronic content comprising the first treatment of the first schema;
receiving a signal comprising updated information for the first user;
parsing the updated information; and
modifying the first schema based on the parsed updated information.

16. The non-transitory computer-readable medium of claim 15, wherein the operations further comprise:
transmitting the updated information to a provider; and
connecting the provider with the first user for real-time communication.

17. The non-transitory computer-readable medium of claim 15, wherein the operations further comprise:
receiving feedback signals from a first user device;
determining a schema success rate based on the feedback signals; and
modifying the plurality of schemas for the first group, based on the feedback signals.

18. The non-transitory computer-readable medium of claim 15, wherein the operations further comprise:
receiving feedback signals from a first user device;
determining a schema success rate based on the feedback signals; and
modifying the first schema for the first user, based on the feedback signals.

19. The non-transitory computer-readable medium of claim 15, wherein identifying the first group for the first user is based on a group machine learning model output.

20. The non-transitory computer-readable medium of claim 15, wherein the schema is identified based on an output of a schema machine learning model output.
